(19) United States
(12) Patent Application Publication
(10) Pub. No.: US 2005/0004622 A1
(43) Pub. Date: Jan. 6, 2005

(54) System and method for implantable pulse generator with multiple treatment protocols

(75) Inventors: Patrick M. Cullen, Dallas, TX (US); John H. Erickson, Plano, TX (US); Galen L. Smith, Allen, TX (US)

Correspondence Address: DOCKET CLERK, DM/ANSI, P.O. BOX 802432, DALLAS, TX, 75380, US

(73) Assignee: ADVANCED NEUROMODULATION SYSTEMS, Plano, TX (US)

(21) Appl. No.: 10/613,142

(22) Filed: Jul. 3, 2003

(51) Int. Cl. A61N 1/18

(52) U.S. Cl. 607/46

(57) ABSTRACT

A system, method, and implantable pulse generator (IPG) device that stores, on the implantable device, two or more treatment-protocol stimulus programs, preferably as prescribed by a physician. The IPG, whether it is a self-contained implantable pulse generator (SCIPG) or externally-powered implantable pulse generator (EPIPG), communicates with an external patient programmer (EPP) to determine which of the stimulus programs should be run at any given time. An advanced programmer is used to read and write program instructions to the IPG. In this way, the patient is capable of carrying two or more program options with him, and if the patient uses an EPIPG, he can use any available EPP to power and operate the EPIPG.

### TECHNICAL FIELD OF THE INVENTION

The present invention is directed, in general, to medical devices and, more specifically, to implantable pain-control devices.

### BACKGROUND OF THE INVENTION

The present invention relates to a spinal cord stimulation system. A spinal cord stimulation system is an implantable pulse generating system used to provide electrical stimulation pulses from an electrode array placed epidurally or surgically near a patient's spine. An implanted pulse generator (IPG) may operate independently to provide the required electrical stimulation, or may interact with an external programmer, which delivers programming and/or control information and/or energy for the electrical stimulation, typically through a radio-frequency (RF) or other wireless signal.

Spinal cord stimulation (SCS) is a well accepted clinical method for reducing pain in certain populations of patients. SCS systems typically include an implanted device, lead wires, and electrodes connected to the lead wires. The implanted device receives signals from an external programmer, and transmits corresponding electrical pulses that are delivered to the spinal cord (or other tissue) through the electrodes which are implanted along the dura of the spinal cord. In a typical situation, the attached lead wires exit the spinal cord and are tunneled around the torso of the patient to a sub-cutaneous pocket where the device is implanted.

Spinal cord and other stimulation systems are known in the art. For example, in U.S. Pat. No. 3,646,940, there is disclosed an implantable electronic stimulator that provides timed sequenced electrical impulses to a plurality of electrodes so that only one electrode has a voltage applied to it at any given time. Thus, the electrical stimuli provided by the apparatus taught in the 1940 patent comprise sequential, or non-overlapping, stimuli.

In U.S. Pat. No. 3,724,467, an electrode implant is disclosed for the neuro-stimulation of the spinal cord. A relatively thin and flexible strip of physiologically inert plastic is provided with a plurality of electrodes formed thereon. The electrodes are connected by leads to an RF receiver, which is also implanted, and which is controlled by an external controller. The implanted RF receiver has no power storage means, and must be coupled to the external controller in order for neuro-stimulation to occur.

In U.S. Pat. No. 3,822,708, another type of electrical spinal cord stimulating device is shown. The device has five aligned electrodes which are positioned longitudinally on the spinal cord and transversely to the nerves entering the spinal cord. Current pulses applied to the electrodes are said to block sensed intractable pain, while allowing passage of other sensations. The stimulation pulses applied to the electrodes are approximately 250 microseconds in width with a repetition rate of from 5 to 200 pulses per second. A patient-operable switch allows the patient to change which electrodes are activated, i.e., which electrodes receive the current stimulus, so that the area between the activated electrodes on the spinal cord can be adjusted, as required, to better block the pain.

Other representative patents that show spinal cord stimulation systems or electrodes include U.S. Pat. Nos. 4,338,945; 4,379,462; 5,121,754; 5,417,719, 5,501,703, and 6,516,227. All of the patents noted above are hereby incorporated by reference.

A typical IPG is self contained, having a multi-year battery pack and a single treatment program, and is generally programmed during or immediately following implantation in the patient's body.

Other SCS systems have no implanted power source, but receive power and programming and/or control information from an external transmitter. These systems will convert the RF signals from the transmitter to provide power to the implanted receiver, and use the RF programming information to determine the intensity, location, and duration of the electrical pulses delivered to the electrodes.

There is a significant programming limitation with known SCS systems. In a typical IPG, the patient's program is installed during implantation, and the patient must visit a doctor to have any programming changes made.

In an externally-powered SCS system, the transmitter carries the patient's programming, which it communicates to the implanted receiver. In order to prevent mistaken use of another, differently-programmed transmitter, the patient's transmitter is effectively “tied” to the patient's receiver for the entire life of the receiver. If the patient should use another transmitter, it will send the receiver a stimulation program that may be inappropriate or even harmful to the patient, a problem that is addressed by using a serial number or other authentication to ensure that only the patient's specific transmitter will interact with his receiver.

Further, since all programming for an SCS receiver is stored on the transmitter, the patient must carry the transmitter with him whenever he requires a change in prescription or programming, since the transmitter itself must be reprogrammed.

There is, therefore, a need in the art for a system, process and device for improved programming options for IPGs.

### SUMMARY OF THE INVENTION

In one embodiment of the present invention, there is a system, method, and implantable pulse generator (IPG) device that stores, on the implantable device, two or more stimulus programs, preferably as prescribed by a doctor. The IPG, whether it is a self-contained implantable pulse generator (SCIPG) or externally-powered implantable pulse generator (EPIPG), communicates with an external patient programmer (EPP) to determine which of the stimulus programs should be run at any given time. An advanced programmer is used to read and write program instructions to the IPG. In this way, the patient is capable of carrying two or more program options with him, and if the patient uses an EPIPG, he can use any available EPP to power and operate the EPIPG.

The foregoing has outlined rather broadly the features and technical advantages of the present invention so that those skilled in the art may better understand the detailed description of the invention that follows. Additional features and advantages of the invention will be described hereinafter that form the subject of the claims of the invention. Those skilled in the art will appreciate that they may readily use the conception and the specific embodiment disclosed as a basis for modifying or designing other structures for carrying out the same purposes of the present invention. Those skilled in the art will also realize that such equivalent constructions do not depart from the spirit and scope of the invention in its broadest form.

### BRIEF DESCRIPTION OF THE DRAWINGS

For a more complete understanding of the present invention, and the advantages thereof, reference is now made to the following descriptions taken in conjunction with the accompanying drawings, wherein like numbers designate like objects, and in which:

FIG. 1 depicts a block diagram of an implantable pulse generator in accordance with a preferred embodiment of the present invention;

FIG. 2 depicts a flowchart of a process in accordance with a preferred embodiment of the present invention; and

FIG. 3 depicts a flowchart of a process in accordance with a preferred embodiment of the present invention.

### DETAILED DESCRIPTION OF THE INVENTION

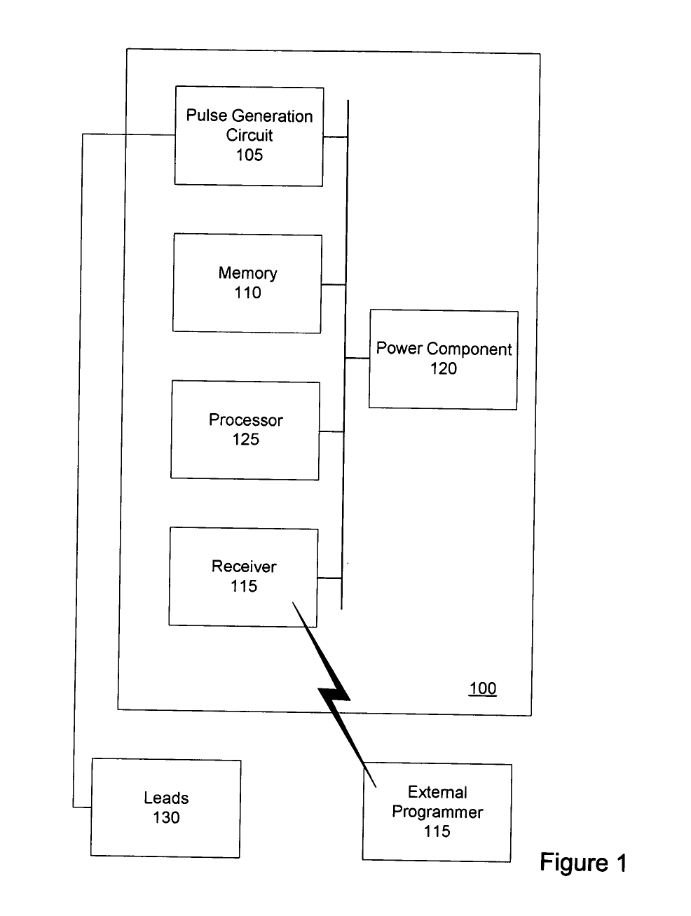

FIGS. 1 through 3, discussed below, and the various embodiments used to describe the principles of the present invention in this patent document are by way of illustration only and should not be construed in any way to limit the scope of the invention. Those skilled in the art will understand that the principles of the present invention may be implemented in any suitably arranged device. The numerous innovative teachings of the present application will be described with particular reference to the presently preferred embodiment.

One embodiment of the present invention provides a system, method, and implantable pulse generator (IPG) device that stores, on the implantable device, two or more stimulus programs, preferably as prescribed by a doctor. The IPG, whether it is a self-contained implantable pulse generator (SCIPG) or externally-powered implantable pulse generator (EPIPG), communicates with an external patient programmer (EPP) to determine which of the stimulus programs should be run at any given time. An advanced programmer is used to read and write program instructions to the IPG. In this way, the patient is capable of carrying two or more program options with him, and if the patient uses an EPIPG, he can use any available EPP to power and operate the EPIPG.

As used herein, an SCIPG is an IPG having an implanted power source, such as a long-lasting or rechargeable battery. An EPIPG is an IPG which receives at least some of its operating power from an external power transmitter, preferably in the form of a RF signal. The external power transmitter, in the preferred embodiment, built into the EPP.

FIG. 1 shows a diagram of the components of an IPG 100 in accordance with the preferred embodiment. The implanted device comprises, but is not limited to, a pulse generation circuit 105, a non-volatile memory 110, a receiver 115, a power component 120, and a processor 125. Memory 110 may also include volatile memory (not shown).

In an SCIPG, the power component 115 will include a long-term battery and a voltage detection and regulation circuit. In an EPIPG, the power component 120 will include a circuit for converting radio-frequency (RF) energy (or other energy) into direct current. In either case, the power component is connected to power the processor 125 and the pulse generation circuit 105.

One example of an SCIPG may be an SCIPG manufactured by Advanced Neuromodulation Systems, Inc. such as the Genesis® system, part number 3608. One example of the EPIPG may be on EPIPG manufactured by Advanced Neuromodulation Systems, Inc. such as the Renew® system, part number 3416.

The pulse generation circuit 105 is connected to receive power from power component 120 and to be controlled by processor 125. Processor 125 is connected to receive power from power component 120 and to read from, and write to, non-volatile memory 110. Further, processor 125 is connected to receive and decode data from receiver 115.

Receiver 115 is positioned to receive RF commands from an external programmer, and to deliver these commands to processor 125. Further, in an EPIPG, the receiver 115 is configured to receive RF power signals, and to deliver these to power component 120.

Non-volatile memory 110 contains programming and control data, and can be written to and read from by processor 125.

Leads 130 are implanted in the patient's epidural space (or other locations), as described above. Leads 130 connect with pulse generation circuit 110, optionally via lead extensions (not shown).

Leads 130, in one embodiment, have multiple electrodes, each of which can be independently controlled by the pulse generation circuit 110. Each electrode can individually biased with a positive pulse (acting as an anode), a negative pulse (acting as a cathode), or turned off. The pulse generation circuit 110, under control of the processor 125, also controls the pulse amplitude, pulse width, and pulse frequency to each electrode on the leads 130.

Also shown here, although not a part of the IPG 100 itself, is external programmer 150, which communicates with receiver 115. External programmer 150 can be either an EPP, which is typically carried and operated by the patient, or an advanced programmer, which is typically operated by the patient's physician. External programmer 150 will typically communicate with receiver 115 via an antenna (not shown), placed on or near the patient's body proximal to the IPG 100.

In a conventional EPIPG, the external programmer is used to send both a power signal and pulse-generation instructions, on a real-time basis, to the EPIPG. In this case, the programming for the EPIPG is stored on the external programmer.

One of the differences between the preferred embodiment and conventional systems is that, in the preferred embodiment, multiple treatment programs are stored on the IPG by using an advanced programmer by the patient's physician or other professional, then the patient can use his external programmer to select between the multiple programs and/or change customizable options such as pulse amplitude parameters. In the case of an EPIPG, the external programmer will also supply a power signal to the IPG.

According to one embodiment, with multiple treatment programs stored on the IPG, the patient can use any compatible external programmer to select between the programs or change options. In this way, unlike in conventional EPIPGs, the patient is not “shackled” to his specific, prescribed external programmer, and can use any available external programmer, such as one at his physician's office, or a spare he might store in his car.

By storing multiple treatment programs, each of which has been prescribed and stored on the IPG by the physician, the patient is able to select the appropriate treatment program for his current activities, using any available compatible external programmer, without having to worry that the programmer will attempt to operate his IPG with a non-prescribed, and potentially harmful, program.

Because, according to one embodiment, all treatment programming is stored on the IPG, the only difference between an SCIPG and an EPIPG, in this case, is whether the power source is also implanted, as in the SCIPG. All treatment programming is stored in the SCIPG, and both types of IPGs allow the programs to be selected using an external programmer. Since the external programmer is no longer required to be “tied” to a specific patient or IPG, any compatible external programmer can be used, including a “universal” external programmer.

A program consists of one or more stimulation settings, also referred to herein as “stimsets.” The programmed stimulation settings specifically define and characterize the administered electric pulse stimulation. Further details of stimulation settings and application, not necessary for an understanding of the presently preferred embodiment, are found in U.S. patent application Ser. No. 08/659,919, filed Jun. 7, 1996, which is hereby incorporated by reference.

In one embodiment, each stimset is comprised of an electrode configuration and stimulation amplitude, stimulation frequency, stimulation pulse width and/or signal phase information. The electrode configuration defines whether each electrode is on or off and, if on, the polarity of that electrode. The amplitude is the intensity of the applied electric pulse. The frequency is the number of times the electrodes are turned on each second. The pulse width is the amount of time the electrodes are left on during each cycle. Finally, the signal phase setting defines the stimulation waveform as “monophasic” (either a positive or negative pulse) or “biphasic” (an alternating negative-positive or positive-negative pulse).

A program is defined as having at least one stimset, and generally corresponds to providing a treatment relating to a specific part of a patient's body. A program can have multiple stimsets; in this case, each stimset is applied sequentially (and repeatedly). Preferably, each sequential stimset is applied quickly enough so that the patient experiences the combined effect of each stimset, as if they were being applied simultaneously.

For example, a first stimset may provide relief to a patient's right leg, and a second stimset may provide relief to a patient's left leg. According to one embodiment, then, there will be at least three programs stored on the patient's IPG:
Program 1 comprises the first stimset;
Program 2 comprises the second stimset; and
Program 3 comprises both the first and second stimsets.

In this case, when the patient uses program 1 on the IPG, she would feel relief in her right leg, program 2 would provide relieve in her left leg, and program 3 would provide relieve in both legs.

In one embodiment, the IPG is capable of storing up to 24 different programs, each program having up to 8 stimsets. Of course, depending on the amount of memory available, the IPG can potentially store a much greater number of programs, each having associated a much greater number of stimsets.

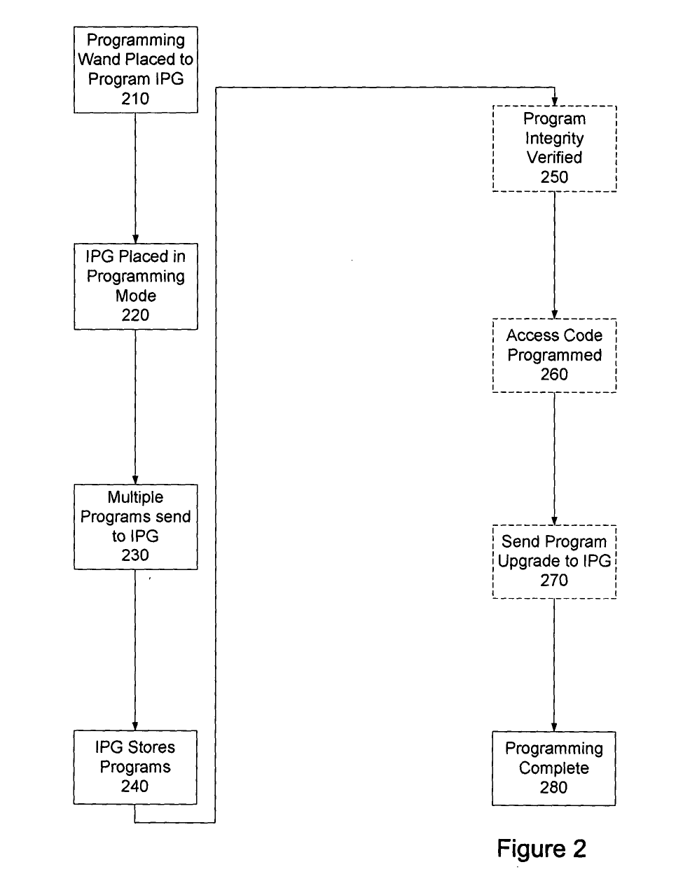

FIG. 2 depicts a flowchart of a process for programming the IPG with multiple treatment-protocol programs. Note that this process is used to program an already-implanted IPG; a similar process can be used to pre-program the IPG before implantation.

This process is typically performed by a physician or other professional using an advanced programmer, as described herein. Generally, this programming process is not one that would normally be performed by a patient, but could be so if the patient were properly trained.

First, a programming wand will be placed in a location proximate to the IPG or the IPG antenna (step 210). In other embodiments, “far-field” programming can be used. Next, preferably using an RF signal, the advanced programmer will place the IPG into programming mode (step 220).

The advanced programmer is then used to send at least two patient-prescribed treatment-protocol programs to the IPG (step 230). The IPG will store these programs in non-volatile memory (step 240).

Optionally, the IPG will then verify correct receipt of the programs using a checksum or other method (step 250), and can receive an access code to restrict access to the treatment protocol programming (step 260). Also, the same programming technique can be used to replace or upgrade the IPG internal programming (step 270).

Programming is then complete (step 280). The IPG is, at this point, programmed with multiple treatment-protocol programs, which can be selected by the patient as described herein.

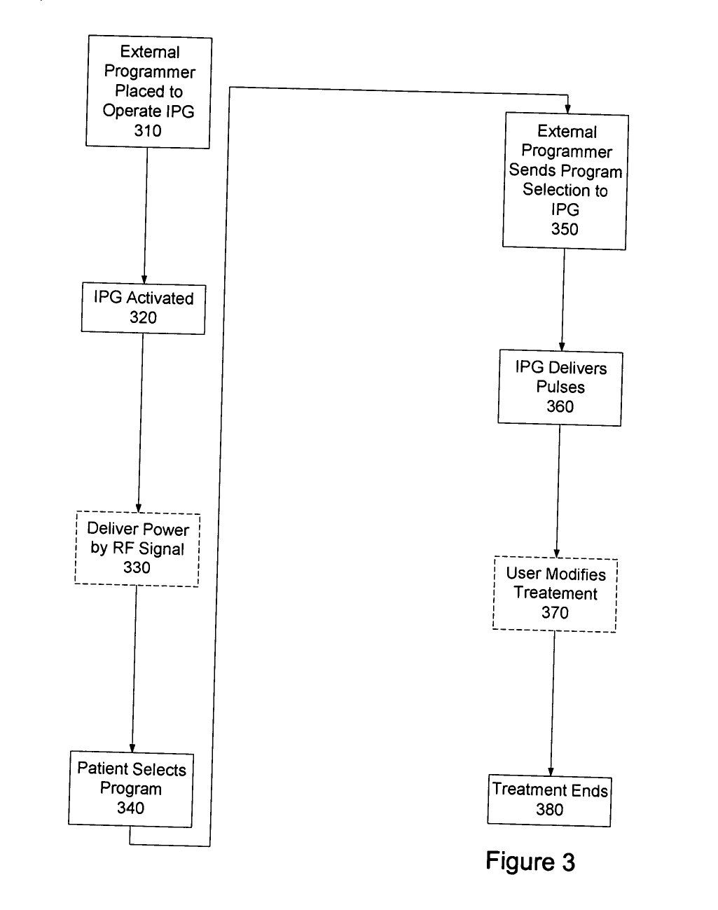

FIG. 3 depicts a flowchart that describes the use of an IPG having multiple treatment-protocol programs stored within. This process is generally performed by the patient.

First, the external programmer will be placed in a location proximate to the IPG or the IPG antenna (step 310). Next, preferably using an RF signal, the advanced programmer will activate the IPG (step 320).

During operation, the external programmer will optionally, as in the case of an EPIPG, supply power to the IPG, preferably using an RF signal (step 330). The patient will select the treatment protocol on the external programmer (step 340), and the external programmer will send an RF signal to the IPG to indicate the selected treatment-protocol program (step 350). Alternately, if a treatment protocol selection is not sent by the external programmer, the IPG will select one of the stored treatment-protocol programs as the “default” program.

The IPG delivers the pulse stimulus, as described herein, according to the selected treatment-protocol program (step 360) and its associated stimsets. Optionally, the user can modify the intensity or other aspects of the treatment as needed, using the external programmer (step 370). For example, a typical modification is to change the intensity setting using the external programmer, causing the IPG to adjust the pulse amplitude delivered to the lead electrodes.

When the treatment program is completed, or when the user chooses, the pulse-stimulus treatment ends (step 380).

Those skilled in the art will recognize that, for simplicity and clarity, the full structure and operation of all devices and processes suitable for use with the present invention is not being depicted or described herein. Instead, only so much of an implantable pulse generator and supporting hardware as is unique to the present invention or necessary for an understanding of the present invention is depicted and described. The remainder of the construction and operation of the IPGs described herein may conform to any of the various current implementations and practices known in the art.

Those of skill in the art will also recognize that not all steps in the above-described processes must be performed in the order described. Further, not all steps of any process, particularly the optional steps, must necessarily be performed in conjunction with all other steps, and can be omitted from the process or performed independent of other steps of the process.

It is important to note that while the present invention has been described in the context of a fully functional system, those skilled in the art will appreciate that at least portions of the mechanism of the present invention are capable of being distributed in the form of an instruction set contained within a machine usable medium in any of a variety of forms, and that the present invention applies equally regardless of the particular type of instruction or signal bearing medium utilized to actually carry out the distribution. Examples of machine usable mediums include: nonvolatile, hard-coded type mediums such as read only memories (ROMs) or erasable, electrically programmable read only memories (EEPROMs), user-recordable type mediums such as floppy disks, hard disk drives and compact disk read only memories (CD-ROMs) or digital versatile disks (DVDs), and transmission type mediums such as digital and analog communication links.

Although an exemplary embodiment of the present invention has been described in detail, those skilled in the art will understand that various changes, substitutions, variations, and improvements of the invention disclosed herein may be made without departing from the spirit and scope of the invention in its broadest form.

None of the description in the present application should be read as implying that any particular element, step, or function is an essential element which must be included in the claim scope: THE SCOPE OF PATENTED SUBJECT MATTER IS DEFINED ONLY BY THE ALLOWED CLAIMS. Moreover, none of these claims are intended to invoke paragraph six of 35 USC §112 unless the exact words “means for” are followed by a participle.

It may be advantageous to set forth definitions of certain words or phrases used throughout this patent document: the terms “include” and “comprise,” as well as derivatives thereof, mean inclusion without limitation; the term “or” is inclusive, meaning and/or; the phrases “associated with” and “associated therewith,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, or the like; and if the term “controller” is utilized herein, it means any device, system or part thereof that controls at least one operation, whether such a device is implemented in hardware, firmware, software or some combination of at least two of the same. It should be noted that the functionality associated with any particular controller may be centralized or distributed, whether locally or remotely.

1. An implantable pulse generator, comprising:
a pulse generation circuit;
a processor connected to control the pulse generation circuit;
a memory connected to the processor, the memory operable to store at least two treatment-protocol programs, each program having at least one stimulation setting, and at least one of the programs having a plurality of stimulation settings; and
a power component configured to supply power to the pulse generation circuit, the processor, and the memory.

2. The implantable pulse generator of claim 1, further comprising a receiver connected to communicate with the processor, the receiver being configured to receive wireless programming signals.

3. The implantable pulse generator of claim 1, wherein the power component comprises a receiver for receiving an externally-generated power signal.

4. The implantable pulse generator of claim 1, wherein the pulse generation circuit is connected to deliver stimulus pulses to epidurally or surgically implanted leads.

5. The implantable pulse generator of claim 1, wherein the treatment-protocol programs are independently selectable.

6. The implantable pulse generator of claim 1, wherein one of the treatment-protocol programs is designated as a default treatment-protocol program.

7. The implantable pulse generator of claim 1, wherein the treatment-protocol programs can be programmed and selected by an external programmer.

8. A method for programming a stimulation device, comprising:
placing an implantable pulse generator in a programming mode using an external programming device; and
sending at least two treatment protocol programs from the external programming device to the implantable pulse generator, wherein the treatment protocol programs are stored in a memory in the implantable pulse generator, and wherein each treatment protocol program is associated with at least one stimulation setting, and at least one of the programs is associated with a plurality of stimulation settings.

9. The method of claim 8, further comprising verifying that the treatment protocol programs were correctly stored.

10. The method of claim 8, further comprising designating an access code for treatment protocol programs.

11. The method of claim 8, further comprising receiving an externally-generated power signal.

12. The method of claim 8, wherein the treatment-protocol programs are independently selectable.

13. The method of claim 8, wherein one of the treatment-protocol programs is designated as a default treatment-protocol program.

14. The method of claim 8, wherein the treatment-protocol programs can be thereafter selected by an external programmer.

15. A method for operating a stimulation device, comprising:
placing an implantable pulse generator in an activated mode using an external programming device; and
sending a program-selection signal to the implantable pulse generator by the external programming device, wherein the implantable pulse generator stores at least two treatment protocol programs, each treatment protocol program being associated with at least one stimulation setting, and at least one of the programs being associated with a plurality of stimulation settings;
thereafter controlling the operation of the implantable pulse generator by the external programming device.

16. The method of claim 15, further comprising delivering a power signal to the implantable pulse generator by the external programming device.

17. The method of claim 15, wherein the external programming device communicates with the implantable pulse generator using a radio-frequency signal.

18. The method of claim 15, wherein the external programming device can control the pulse amplitude parameters of the pulses generated by the implantable pulse generator.

19. The method of claim 15, wherein the program selection signal designates which of the treatment protocol programs is to be executed by the implantable pulse generator.

20. The method of claim 15, wherein the external programming device is operated by a patient in whom the implantable pulse generator is implanted.
